(19) United States
(12) Patent Application Publication
(10) Pub. No.: US 2013/0345077 A1
(43) Pub. Date: Dec. 26, 2013

(54) DIAGNOSIS OF LYMPH NODE INVOLVEMENT IN RECTAL CANCER

(71) Applicant: THE CLEVELAND CLINIC FOUNDATION, Cleveland, OH (US)

(72) Inventors: Matthew F. Kalady, Cleveland Heights, OH (US); J. Calvin Coffey, Limerick (IE)

(21) Appl. No.: 13/864,589

(22) Filed: Apr. 17, 2013

Related U.S. Application Data
(60) Provisional application No. 61/625,388, filed on Apr. 17, 2012.

(51) Int. Cl. C12Q 1/68

(52) U.S. Cl. CPC C12Q 1/6886
USPC 506/9; 506/16

(57) ABSTRACT

A method of determining the risk that a subject that has been diagnosed with rectal cancer has stage III rectal cancer is described. The method includes determining the expression levels of a plurality of differentially expressed genes in a rectal cancer sample from the subject, comparing the expression levels of the plurality of genes with the corresponding controls, and characterizing the subject as having an increased risk of having stage III rectal cancer if the expression levels of the genes are increased or decreased compared to the corresponding control values. The inventor has determined which genes are expressed at higher or lower levels by a subject who has stage III rectal cancer. Microarrays and kits for staging subjects are also provided.

### CONTINUING APPLICATION DATA

This application claims the benefit of U.S. Provisional Patent Application No. 61/625,388, filed Apr. 17, 2012, which is incorporated by reference herein.

### BACKGROUND

Accurate diagnosis of locoregional lymph node involvement is paramount in the treatment of rectal cancer. The spread of cancer from the primary tumor to lymph nodes dramatically worsens prognosis and affects treatment decision making. Suspicion of lymph node positivity is an indication for neoadjuvant chemoradiation and adjuvant chemotherapy. Furthermore, the clinical diagnosis of lymph node involvement or the risk of lymph node involvement drives more aggressive surgery such as protectomy as opposed to less invasive and less morbid local excision.

Unfortunately, diagnosis of metastatic rectal cancer in lymph nodes suffers from inaccuracy both in clinical and pathological staging. Nicastri et al., J Mol Diagn., 9, 563-571 (2007). Thus, treatment decisions are made on the basis of limited, often inaccurate information which can result in either undertreatment or excessive treatment. Clearly, improved diagnostics are needed, and an objective means to preoperatively diagnose lymph node involvement could considerably help individualize treatment algorithms.

Molecular and genetic approaches have been used for individual markers in the primary tumor, but they have not been useful in predicting pathological detection of cancer cells spread to lymph nodes. Zauber et al., J Clin Pathol., 57, 938-942 (2004). Development and progression of cancer is a complex process involving myriad genetic and epigenetic changes. Therefore, it is not surprising that 1 individual gene or protein does not serve as an effective biomarker. cDNA microarray technology has allowed for a broader approach to finding genetic and molecular biomarkers and signatures. This technology is now routinely used in the research laboratory and has provided insight into cancer biology.

Gene signatures have been used in colorectal cancer to define biological pathways, describe differences in treatment response, and predict outcomes. Kalady et al., J Am Coll Surg., 211, 187-195 (2010); Watanabe et al., Cancer, 115, 283-292 (2009). Gene expression studies have also been used to develop signatures that predict lymph node involvement in colorectal cancer. Watanabe et al., Dis Colon Rectum., 52, 1941-1948 (2009). These studies are hindered by small sample sizes and the combined analysis of both colon and rectal cancers. The vast majority of cases in these studies are colon cancers and no study has specifically addressed rectal cancers alone. Because there are biological differences between colon and rectal cancers (Kalady et al., Dis Colon Rectum., 52, 1039-1045 (2009)) it is important to define signatures specifically from a pure rectal cancer population.

### SUMMARY OF THE INVENTION

The inventors have determined that distinct gene expression signatures from primary rectal adenocarcinomas can help differentiate the presence or absence of lymph node metastases, and that this may provide an improved approach for individualized treatment selection.

Accordingly, in one aspect, the invention provides a method of determining the risk that a subject that has been diagnosed with rectal cancer has stage III rectal cancer that includes the steps of determining the expression levels of a plurality of differentially expressed genes selected from table 1a and/or table 1b in a rectal cancer sample from the subject, comparing the expression levels of the plurality of genes with the corresponding controls, and characterizing the subject as having an increased risk of having stage III rectal cancer if the expression levels of the genes from table 1a are increased compared to the corresponding control values, and/or the expression levels of the genes from table 1b are decreased compared to the corresponding control values. In some embodiments, the subject has been previously diagnosed as having stage II rectal cancer.

In one embodiment, the plurality of differentially expressed genes each show an at least ±0.5 fold change relative to the corresponding control values. In another embodiment, the one or more of the differentially expressed genes are selected from the group consisting of SSBP1, HMGCS2, CEL, CST1, LY6G6D, SNAR-A1, DES, PCP4, ACTG2, and MYH11. In a further embodiment the one or more of the differentially expressed genes are selected from the group consisting of REG4, CA1, GCNT3, ITLN1, IL8, HLA-DRB1, LOC652775, SPINK4, CLCA1, and LYZ. In a yet further embodiment, the one or more differentially expressed genes are selected from the group consisting of genes for interleukin-8,3-hydroxy-3-methylglutaryl coenzyme A synthase, carbonic anhydrase, ubiquitin, and cystatin, or include all of these genes. In some embodiments, the expression levels of at least 50 differentially expressed genes are determined.

In another embodiment, the plurality of differentially expressed genes are part of a network of genes based on tumor necrosis factor. In further embodiments, the differentially expressed genes are genes known to be functionally associated with cancer, gastrointestinal disease, or cellular movement, or are known to be part of immune-related pathways.

In yet further embodiments, the method includes the step of extracting RNA from the rectal cancer sample before determining the expression levels of a plurality of differentially expressed genes. In additional embodiments, the method includes the step of treating or recommending treatment of a subject identified as having an increased risk of having progressed to stage III with anticancer therapy suitable for treatment of stage III rectal cancer.

Another aspect of the invention provides a microarray for determining the risk that a subject diagnosed with rectal cancer has progressed to stage III rectal cancer. The microarray includes a plurality, or in some embodiments at least 25 polynucleotide probes having polynucleotide sequences complementary to the polynucleotide sequence of the corresponding differentially expressed genes from table 1a and/or table 1b. In additional embodiments, the polynucleotide probes include polynucleotide sequences complementary to a polynucleotide sequence expressed by the genes for interleukin-8,3-hydroxy-3-methylglutaryl coenzyme A synthase, carbonic anhydrase, ubiquitin, and cystatin.

Another aspect of the invention provides a kit for determining the risk that a subject diagnosed with rectal cancer has progressed to stage III. In some embodiments, the kit is the microarray. In additional embodiments, the kit can include controls for the differentially expressed genes, instructions for using the kit to determining the risk that a subject diagnosed with rectal cancer has progressed to stage III, and/or reagents for amplification of nucleic acids and detectable labels.

### BRIEF DESCRIPTION OF THE FIGURES

The present invention may be more readily understood by reference to the following figures, wherein:

FIG. 1 provides a dendrogram of stage II and stage III samples by the use of normalized data with Pearson correlation and average linkage method. A proportion of stage III samples cluster centrally, as indicated with the remaining stage III samples distributed evenly on either side.

FIG. 2 provides a heat map and dendrograms generated by use of Pearson correlation and average linkage. Rectal cancer samples are across the horizontal axis, with 1 sample expression pattern shown in each column. Dendrograms demonstrate clustering of related genes or samples. The top horizontal dendrogram shows the clustering of related samples according to gene expression patterns. The left vertical axis dendrogram shows relatedness of genes that tend to cluster together. When the heat map is partitioned according to sample and gene clusters, 4 genes (i.e., interleukin-8, HMG-CoA synthase, carbonic anhydrase, cystatin, and ubiquitin) cluster with a high level of expression in the sample cluster comprising mainly stage III tumors. HMG-CoA=3-hydroxy-3-methylglutaryl coenzyme A.

FIG. 3 provides a graph showing the results of Ingenuity functional analysis. The top 15 functional categories of the top 147 most differentially expressed genes are depicted here. The blue bars represent the number of genes from the data set that are associated with each biological function and/or disease category. The biological functions and/or disease are arranged according to the most significant p value in a descending manner. The x axis is labeled as the negative log of the p value by convention in Ingenuity and represents a score that is used to rank gene networks according to their relevance to the study data set.

FIG. 4 provides a graph showing the results of Ingenuity canonical pathways. The top 147 differentially expressed genes were analyzed. The blue bars represent the number of molecules associated with the top 15 most frequently represented canonical pathway. The ratio represents the number of molecules associated with the canonical pathway in the current data set vs. the total number of molecules assigned to that pathway in the database. The canonical pathways are arranged based on p value. The x axis is labeled as the negative log of the p value by convention in Ingenuity and represents a score which is used to rank gene networks according to their relevance to the study data set.

FIG. 5 provides a scheme showing the Results of Ingenuity network analysis. The five most differentially expressed genes were analyzed by Ingenuity Network analysis. Four of the five top differentially expressed genes, marked by gray, were mapped onto the same network that was centered on TNF. CA1=carbonic anhydrase; IL8=interleukin 8; HMGCOS2=3-hydroxy-3-methylglutaryl coenzyme A synthase; TNF=tumor necrosis factor; UBD=ubiquitin.

### DETAILED DESCRIPTION OF THE INVENTION

The present invention relates to the field of cancer. More specifically, it relates to markers and methods for determining whether a subject that has been diagnosed with rectal cancer, particularly a human subject, has progressed to stage III cancer by determining the expression levels of a plurality of differentially expressed genes. In particular, the use of a microarray to provide a genetic profile to differentiate the presence or absence of lymph node metastases is disclosed.

### DEFINITIONS

As used herein, the term “diagnosis” can encompass determining the likelihood that a subject will develop a disease, or the existence or nature of disease in a subject. The term diagnosis, as used herein also encompasses determining the severity and probable outcome of disease or episode of disease or prospect of recovery, which is generally referred to as prognosis). “Diagnosis” can also encompass diagnosis in the context of rational therapy, in which the diagnosis guides therapy, including initial selection of therapy, modification of therapy (e.g., adjustment of dose or dosage regimen), and the like.

As used herein, the terms “treatment,” “treating,” and the like, refer to obtaining a desired pharmacologic or physiologic effect. The effect may be therapeutic in terms of a partial or complete cure for a disease or an adverse effect attributable to the disease. “Treatment,” as used herein, covers any treatment of a disease in a mammal, particularly in a human, and can include inhibiting the disease or condition, i.e., arresting its development; and relieving the disease, i.e., causing regression of the disease.

Prevention or prophylaxis, as used herein, refers to preventing the disease or a symptom of a disease from occurring in a subject which may be predisposed to the disease but has not yet been diagnosed as having it (e.g., including diseases that may be associated with or caused by a primary disease). Prevention may include completely or partially preventing a disease or symptom.

The terms “individual,” “subject,” and “patient” are used interchangeably herein irrespective of whether the subject has or is currently undergoing any form of treatment. As used herein, the term “subject” generally refers to any vertebrate, including, but not limited to a mammal. Examples of mammals including primates, including simians and humans, equines (e.g., horses), canines (e.g., dogs), felines, various domesticated livestock (e.g., ungulates, such as swine, pigs, goats, sheep, and the like), as well as domesticated pets (e.g., cats, hamsters, mice, and guinea pigs).

The term “gene,” as used herein, refers to a stretch of DNA that codes for a polypeptide or for an RNA chain that has a known function. While it is the exon region of a gene that is transcribed to form RNA (e.g., mRNA), the term “gene,” as used herein, also includes the regulatory regions such as promoters and enhancers that govern expression of the exon region.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs.

As used herein and in the appended claims, the singular forms “a”, “and”, and “the” include plural referents unless the context clearly dictates otherwise. Thus, for example, reference to “a sample” also includes a plurality of such.

Unless otherwise indicated, all numbers expressing quantities of ingredients, properties such as molecular weight, reaction conditions, and so forth as used in the specification and claims are to be understood as being modified in all instances by the term “about.” Accordingly, unless otherwise indicated, the numerical properties set forth in the following specification and claims are approximations that may vary depending on the desired properties sought to be obtained in embodiments of the present invention. Notwithstanding that the numerical ranges and parameters setting forth the broad scope of the invention are approximations, the numerical values set forth in the specific examples are reported as precisely as possible. Any numerical values; however, inherently contain certain errors necessarily resulting from error found in their respective measurements.

### Diagnostic Methods

The present disclosure provides a method of determining the risk that a subject diagnosed with rectal cancer has progressed to stage III rectal cancer. The method includes determining the expression levels of a plurality of differentially expressed genes in a rectal cancer sample from the subject, comparing the expression levels of the plurality of genes with the corresponding controls, and characterizing the subject as having an increased risk of having progressed to stage III if the expression levels of the genes are increased or decreased compared to the corresponding control values. The differentially expressed genes have been identified by the inventors, and are described herein. In addition, whether these differentially expressed genes show higher or lower expression in subjects having stage III rectal cancer is also described.

The differentially expressed genes that can be used to determine the risk that a subject has progressed to stage III rectal cancer were identified by the inventors as the result of microarray analysis of rectal cancer samples, and are described in Table 1. The differentially expressed genes are genes that were either expressed at a higher level or a lower level in subjects who had progressed to stage III rectal cancer, in comparison with the levels seen in subjects who had not progressed to stage III rectal cancer. The differentially expressed genes provided in table 1a are genes that showed a lower level of expression in stage III tumors, whereas the differentially expressed genes provided in table 1b are genes that showed an increased level of expression in stage III tumors.

TABLE 1a
Top differentially expressed genes between the sample clusters based on unsupervised hierarchical clustering

| Probe ID | P | Fold Change | Symbol | Entrez Gene Name |
|---|---|---|---|---|
| ILMN_1809478 | 0.0001 | −3.439 | SSBP1 | Single-stranded DNA binding protein 1 |
| ILMN_1815203 | 0.0091 | −1.347 | HMGCS2 | 3-Hydroxy-3-methylglutaryl-CoA synthase 2 (mitochondrial) |
| ILMN_1723418 | 0.0076 | −1.323 | CEL | Carboxyl ester lipase (bile salt-stimulated lipase) |
| ILMN_1753449 | 0.0027 | −1.303 | CST1 | Cystatin SN |
| ILMN_1696295 | 0.0159 | −1.238 | LY6G6D | Lymphocyte antigen 6 complex, locus G6D |
| ILMN_1881909 | 0.0142 | −1.201 | SNAR-A1 | Small ILF3/NF90-associated RNA A1 |
| ILMN_1698995 | 0.0047 | −1.185 | DES | Desmin |
| ILMN_1682326 | 0.0027 | −1.171 | PCP4 | Purkinje cell protein 4 |
| ILMN_1795325 | 0.0012 | −1.169 | ACTG2 | Actin, γ 2, smooth muscle, enteric |
| ILMN_1660086 | 0.0055 | −1.094 | MYH11 | Myosin, heavy chain 11, smooth muscle |
| ILMN_1804922 | 0.0507 | −1.068 | IGF2 | Insulin-like growth factor 2 (somatomedin A) |
| ILMN_1736078 | 0.0160 | −0.990 | THBS4 | Thrombospondin 4 |
| ILMN_1759089 | 0.0619 | −0.970 | DEFA6 | Defensin, α 6, Paneth cell-specific |
| ILMN_1770424 | 0.0561 | −0.951 | DEFA5 | Defensin, α 5, Paneth cell-specific |
| ILMN_1712075 | 0.0112 | −0.930 | SYNM | Synemin, intermediate filament protein |
| ILMN_1671478 | 0.0363 | −0.858 | CKB | Creatine kinase, brain |
| ILMN_1783142 | 0.1383 | −0.849 | RPS4Y1 | Ribosomal protein S4, Y-linked 1 |
| ILMN_1794638 | 0.0303 | −0.849 | VIP | Vasoactive intestinal peptide |
| ILMN_1678841 | 0.0284 | −0.848 | UBD | Ubiquitin D |
| ILMN_1801832 | 0.1901 | −0.743 | PRAC | Prostate cancer susceptibility candidate |
| ILMN_1722898 | 0.1247 | −0.721 | SFRP2 | Secreted frizzled-related protein 2 |
| ILMN_1660317 | 0.1485 | −0.701 | LEFTY1 | Left-right determination factor 1 |
| ILMN_1725139 | 0.0581 | −0.675 | CA9 | Carbonic anhydrase IX |
| ILMN_1803862 | 0.2507 | −0.665 | LCN15 | Lipocalin 15 |
| ILMN_1677636 | 0.0820 | −0.656 | COMP | Cartilage oligomeric matrix protein |
| ILMN_1784459 | 0.1342 | −0.648 | MMP3 | Matrix metallopeptidase 3 (stromelysin 1, progelatinase) |
| ILMN_1651958 | 0.1039 | −0.608 | MGP | Matrix Gla protein |
| ILMN_1802441 | 0.3580 | −0.603 | REG1A | Regenerating islet-derived 1α |
| ILMN_1681462 | 0.3223 | −0.591 | REG1B | Regenerating islet-derived 1β |
| ILMN_1678947 | 0.0980 | −0.583 | CELP | Carboxyl ester lipase pseudogene |
| ILMN_1804151 | 0.1007 | −0.574 | C3 | Complement component 3 |
| ILMN_1670779 | 0.1528 | −0.537 | DPEP1 | Dipeptidase 1(renal) |
| ILMN_1745356 | 0.1329 | −0.532 | CXCL9 | Chemokine (C-X-C motif) ligand 9 |
| ILMN_1729212 | 0.1507 | −0.515 | GRM8 | Glutamate receptor, metabotropic 8 |
| ILMN_1787266 | 0.2402 | −0.460 | SPINK1 | Serine peptidase inhibitor, Kazal type 1 |
| ILMN_1720433 | 0.2284 | −0.456 | FAM3D | Family with sequence similarity 3, member D |
| ILMN_1651354 | 0.3315 | −0.455 | SPP1 | Secreted phosphoprotein 1 |
| ILMN_1772328 | 0.2995 | −0.438 | FABP1 | Fatty acid binding protein 1, liver |
| ILMN_1755537 | 0.3763 | −0.427 | EIF1AY | Eukaryotic translation initiation factor 1A, Y-linked |
| ILMN_1752965 | 0.2422 | −0.416 | GRM1 | Gremlin 1 |
| ILMN_1789007 | 0.2425 | −0.408 | APOC1 | Apolipoprotein C-1 |
| ILMN_1802192 | 0.3288 | −0.403 | C10orf99 | Chromosome 10 open reading frame 99 |
| ILMN_1720113 | 0.2885 | −0.389 | PTPRO | Protein tyrosine phosphatase, receptor type, O |
| ILMN_1805410 | 0.3197 | −0.374 | C15orf48 | Chromosome 15 open reading frame 48 |
| ILMN_1771482 | 0.3102 | −0.363 | KIAA1324 | KIAA1324 |
| ILMN_1680874 | 0.4134 | −0.325 | TUBB2B | Tubulin, β 2B |
| ILMN_1710875 | 0.3904 | −0.311 | MYEOV | Myeloma overexpressed (in a subset of t(11; 14) positive multiple |
| | | | | myelomas) |
| ILMN_1740938 | 0.4287 | −0.308 | APOE | Apolipoprotein E |
| ILMN_1791759 | 0.4057 | −0.291 | CXCL10 | Chemokine (C-X-C motif) ligand 10) |
| ILMN_1740586 | 0.4581 | −0.288 | PLA2G2A | Phospholipase A2, group IIA (platelets, synovial fluid) |
| ILMN_1672776 | 0.4607 | −0.278 | COL10A1 | Collagen, type X, α 1 |
| ILMN_1709139 | 0.5966 | −0.260 | BGN | Biglycan |
| ILMN_1730706 | 0.4763 | −0.249 | FOSB | FBJ murine osteosarcoma viral oncogene homolog B |
| ILMN_1659984 | 0.5378 | −0.237 | MEP1A | Meprin A, α (PABA peptide hydrolase) |
| ILMN_1810172 | 0.5196 | −0.236 | SFRP4 | Secreted frizzled-related protein 4 |
| ILMN_1739582 | 0.5399 | −0.223 | HOXA9 | Homeobox A9 |
| ILMN_1786720 | 0.5943 | −0.208 | PROM1 | Prominin 1 |
| ILMN_1685608 | 0.6595 | −0.186 | NPTX2 | Neuronal pentraxin II |
| ILMN_1709348 | 0.6008 | −0.183 | ALDH1A1 | Aldehyde dehydrogenase 1 family, member A1 |
| ILMN_1793593 | 0.6314 | −0.179 | FZD10 | Frizzled homolog 10 (Drosophila) |
| ILMN_1744951 | 0.6961 | −0.165 | H19 | H19, imprinted maternally expressed transcript (nonprotein |
| | | | | coding) |
| ILMN_1652199 | 0.663 | −0.165 | LOC642113 | LOC642113 lg κ chain V-1 region HK101 precursor |
| ILMN_1685387 | 0.7891 | −0.159 | PIGR | Polymeric immunoglobulin receptor |
| ILMN_1761946 | 0.7403 | −0.116 | PROM2 | Prominin 2 |
| ILMN_1798496 | 0.7702 | −0.111 | HOXB8 | Homeobox B8 |
| ILMN_1699214 | 0.8776 | −0.087 | LOC647450 | LOC647450 similar to lg κ chain V-1 region HK101 precursor |
| ILMN_1669046 | 0.8199 | −0.083 | FOXQ1 | Forkhead box Q1 |
| ILMN_1715401 | 0.8399 | −0.074 | MTIG | Metallothionein 1G |
| ILMN_1692223 | 0.8748 | −0.066 | LCN2 | Lipocalin 2 |
| ILMN_1739508 | 0.9163 | −0.061 | LOC652493 | lg κ chain V-1 HK102-like |
| ILMN_1772218 | 0.8679 | −0.058 | HLA-DPA1 | Major histocompatibility complex, class II, DP α 1 |
| ILMN_1790529 | 0.8827 | −0.02 | LUM | Lumican |
| ILMN_1795190 | 0.9297 | −0.037 | CLDN2 | Claudin 2 |
| ILMN_1685403 | 0.9605 | −0.022 | MMP7 | Matrix metallopeptidase 7 (matrilysin, uterine) |
| ILMN_1760087 | 0.9722 | −0.018 | SLC26A3 | Solute carrier family 26, member 3 |
| ILMN_1771919 | 0.9810 | −0.013 | LOC652694 | Similar to lg κ chain V-1 region KH102 precursor |

TABLE 1b
Top differentially expressed genes between the sample clusters based on unsupervised hierarchical clustering

| Probe IB | P | Fold Change | Symbol | Entrez Gene Name |
|---|---|---|---|---|
| ILMN_1666845 | 0.9909 | 0.004 | KRT17 | Keratin 17 |
| ILMN_1696339 | 0.9697 | 0.014 | ZIC2 | Zic family member 2 (off-paired homolog, Drosophila) |
| ILMN_1686573 | 0.8671 | 0.060 | DEFB1 | Defensin, β 1 |
| ILMN_1726448 | 0.872 | 0.062 | MMP1 | Matrix metallopeptidase 1 (interstitial collagenase) |
| ILMN_1697499 | 0.8796 | 0.077 | HLA-DRB5 | Major histocompatibility complex, class II, DR β 5 |
| ILMN_1799020 | 0.8441 | 0.077 | MUC12 | Mucin 12, cell surface associated |
| ILMN_1689655 | 0.8045 | 0.091 | HLA-DRA | Major histocompatibility complex, class II, DR α |
| ILMN_1679194 | 0.7946 | 0.091 | UGT2B7 | UDP glucuronosyltransferase 2 family, polypeptide B7 |
| ILMN_1755897 | 0..7821 | 0.096 | UGT2B7 | UDP glucuronosyltransferase 2 family, polypeptide B7 |
| ILMN_1696245 | 0.8371 | 0.104 | IGJ | Immunoglobulin J polypeptide, linker protein for |
| | | | | immunoglobulin α and μ polypeptides |
| ILMN_1801205 | 0.7537 | 0.113 | GPNMB | Glycoprotein (transmembrane) nmb |
| ILMN_1681260 | 0.7586 | 0.113 | LOC643272 | LOC643272 hypothetical protein LOC643272 |
| ILMN_1730054 | 0.7378 | 0.125 | GSTT1 | Glutathione S-transferase θ 1 |
| ILMN_1653026 | 0.7281 | 0.130 | PLAC8 | Placenta-specific 8 |
| ILMN_1791711 | 0.6921 | 0.145 | DUOXA2 | Dual oxidase maturation factor 2 |
| ILMN_1792404 | 0.6728 | 0.147 | TM4SF4 | Transmembrane 4 L 6 family member 4 |
| ILMN_1804357 | 0.6907 | 0.149 | GNG4 | Guanine nucleotide binding protein (G protein), γ 4 |
| ILMN_1695631 | .6601 | 0.156 | CHP2 | Calcineurin B homologous protein 2 |
| ILMN_1725193 | 0.5803 | 0.207 | IGFBP2 | Insulin-like growth factor binding protein 2, 36 kDa |
| ILMN_1733998 | 0.5423 | 0.216 | DHRS9 | Dehydrogenase/reductase (SDR family) member 9 |
| ILMN_1806386 | 0.5870 | 0.229 | FAM55D | Family with sequence similarity 55, member D |
| ILMN_1721354 | 0.5321 | 0.237 | KRT6B | Keratin 6B |
| ILMN_1792748 | 0.5543 | 0.248 | CPS1 | Carbamoyl-phosphate synthase 1, mitochondrial |
| ILMN_1768469 | 0.5740 | 0.252 | TCN1 | Transcobalamin I (vitamin B₁₂ binding protein, R binder family) |
| ILMN_1722489 | 0.5442 | 0.260 | TFF1 | Trefoil factor 1 |
| ILMN_1674228 | 0.5446 | 0.266 | LOC651751 | LOC651751 similar to lg κ chain V-II region RPMI 6410 |
| | | | | precursor |
| ILMN_1763196 | 0.5115 | 0.270 | WDR72 | WD repeat domain 72 |
| ILMN_1680757 | 0.4815 | 0.271 | LRRC26 | Leucine-rich repeat containing 26 |
| ILMN_1660041 | 0.5530 | 0.273 | CEACAM7 | CEA-related cell adhesion molecule 7 |
| ILMN_1808245 | 0.4540 | 0.273 | C8orf84 | Chromosome 8 open reading frame 84 |
| ILMN_1695924 | 0.4592 | 0.282 | KLK11 | Kallikrein-related peptidase 11 |
| ILMN_1799887 | 0.4089 | 0.287 | CTSE | Cathepsin E |
| ILMN_1696584 | 0.4131 | 0.290 | ORM1/ORM2 | Orosomucoid 1 |
| ILMN_1764266 | 0.5012 | 0.291 | CKMT2 | Creatine kinase, mitochondrial 2 (sarcomeric) |
| ILMN_1771970 | 0.4427 | 0.292 | ALDOB | Aldolase B, fructose-bisphosphate |
| ILMN_1728787 | 0.4381 | 0.321 | AGR3 | Anterior gradient homolog 3 (Xenopus laevis) |
| ILMN_1804601 | 0.5229 | 0.330 | LOC649923 | LOC649923 similar to lg γ -2 chain C region |
| ILMN_1808405 | 0.3630 | 0.345 | HLA-DQA1 | Major histocompatibility complex, class II, DQ α 1 |
| ILMN_1752592 | 0.3580 | 0.352 | HLA-DRB4 | Major histocompatibility complex, class II, DR β 4 |
| ILMN_1793888 | 0.3426 | 0.371 | SERPINB5 | Serpin peptidase inhibitor, clade B (ovalbumin), member 5 |
| ILMN_1808677 | 0.2996 | 0.382 | UGT2B17 | UDP glucuronosyltransferase 2 family polypeptide B17 |
| ILMN_1741566 | 0.2898 | 0.394 | BMP7 | Bone morphogenetic protein 7 |
| ILMN_1766650 | 0.2565 | 0.421 | FOXA1 | Forkhead box A1 |
| ILMN_1724375 | 0.3813 | 0.422 | MUC17 | Mucin 17, cell surface associated |
| ILMN_1740717 | 0.2217 | 0.430 | ADH1C | Alcohol dehydrogenase 1C (class I), γ polypeptide |
| ILMN_1693192 | 0.3259 | 0.452 | P13 | Peptidase inhibitor 3, skin-derived |
| ILMN_1774570 | 0.2597 | 0.453 | HLA-DRB5 | HLA-DRB5 major histocompatibility complex, class II, DR β 5 |
| ILMN_1666536 | 0.2468 | 0.465 | VSIG2 | V-set and immunoglobulin domain containing 2 |
| ILMN_1651282 | 0.1915 | 0.468 | COL17A1 | Collagen, type XVII, α 1 |
| ILMN_1743797 | 0.2093 | 0.473 | LOC652102 | Similar to lg heavy chain V-I region HG3 precursor |
| ILMN_1768227 | 0.1899 | 0.477 | DCN | Decorin |
| ILMN_1753954 | 0.3287 | 0.480 | OLFM4 | Olfactomedin 4 |
| ILMN_1699704 | 0.2835 | .491 | MSLN | Mesothelin |
| ILMN_1764309 | 0.1652 | 0.512 | ADH1A | Alcohol dehydrogenase 1A (class I), α polypeptide |
| ILMN_1739390 | 0.2315 | 0.512 | ZG16 | Zymogen granule protein 16 homolog (rat) |
| ILMN_1698659 | 0.1449 | 0.534 | ST6GALNAC1 | ST6 (α-N-acetyl-neuraminyl-2,3-β-galactosyl-1,3)-N- |
| | | | | acetylgalactosaminide α-2,6-sialyltransferase 1 |
| ILMN_1791545 | 0.1304 | 0.547 | KRT23 | Keratin 23 (histone deacetylase inducible) |
| ILMN_1718984 | 0.2259 | 0.548 | FCGBP | Fc fragment of lgG binding protein |
| ILMN_1690223 | 0.0901 | 0.601 | CNTNAP2 | Contactin-associated protein-like 2 |
| ILMN_1780255 | 0.1095 | 0.637 | KLK6 | Kallikrein-related peptidase 6 |
| ILMN_1695157 | 0.1299 | 0.671 | CA4 | Carbonic anhydrase IV |
| ILMN_1728075 | 0.0885 | 0.474 | REG4 | Regenerating islet-derived family, member 4 |
| ILMN_1652431 | 0.0371 | 0.752 | CA1 | Carbonic anhydrase I |
| ILMN_1712082 | 0.0625 | 0.759 | GCNT3 | Glucosaminyl (N-acetyl) transferase 3, mucin type |
| ILMN_1699996 | 0.0522 | 0.787 | ITLN1 | Intelectin 1 (galactofuranose binding) |
| ILMN_1666733 | .0309 | 0.810 | IL8 | Interleukin 8 |
| ILMN_1715169 | 0.1251 | 0.817 | HLA-DRB1 | Major histocompatibility complex, class II, DR β 1 |
| ILMN_1695891 | 0.0372 | 0.819 | LOC652775 | LOC652775 similar to lg κ chain V-V region L7 precursor |
| ILMN_1681263 | 0.1212 | 0.850 | SPINK4 | Serine peptidase inhibitor, Kazal type 4 |
| ILMN_1797219 | 0.0658 | 0.852 | CLCA1 | Chloride channel accessory 1 |
| ILMN_1815205 | 0.0110 | 1.169 | LYZ | Lysozyme |

The method includes determining a change in expression level for a plurality of the differentially expressed genes. Expressed levels are determined by evaluating the levels of products from the gene, such as mRNA or proteins. Since 147 differentially expressed genes have been identified, the method is therefore directed to determining a change in expression level for from 2 to 147 differentially expressed genes, although the method can further comprise the identification of additional genes (either differentially expressed or not). The method is therefore directed to determining at least 2 to 147 differentially expressed genes, including any of the number within this range. For example, in various embodiments, the expression of at least 25, 50, 75, or 100 differentially expressed genes can be determined.

In further embodiments, the method can involve the identification of differentially expressed genes in particular categories. For instance, in some embodiments, it may be preferable to determine the expression levels of differentially expressed genes that show particular levels of change in expression level in comparison with the control levels. For example, it may be preferable to determine the expression levels of genes that have shown a ±0.25 fold change, a ±0.5 fold change, a ±0.75 fold change, or a ±1.0 fold change relative to the corresponding control values.

In further embodiments, it may be preferable to determine the expression levels of specific differentially expressed genes. For example, in some embodiments, it may be preferable to determine the expression level of genes showing decreased expression in subjects that have progressed to stage III rectal cancer (i.e., the genes shown in Table 1a). For example, in one embodiment, the one or more differentially expressed genes can be selected from the group consisting of SSBP1, HMGCS2, CEL, CST1, LY6G6D, SNAR-A 1, DES, PCP4, ACTG2, and MYH11, IGF2, THBS4, DEFA6, DEFA5, SYNM, CKB, RPS4Y1, VIP, UBD, and PRAC, while in another embodiment the one or more of the differentially expressed genes are selected from the group consisting of SSBP1, HMGCS2, CEL, CST1, LY6G6D, SNAR-A1, DES, PCP4, ACTG2, and MYH11. The genes are listed here by their abbreviations; the full names for the genes are provided in Table 1.

In other embodiments, it may be preferable to determine the expression level of genes showing increased expression in subjects who have progressed to stage III rectal cancer (i.e., the genes shown in Table 1b). For example, in one embodiment, the one or more differentially expressed genes can be selected from the group consisting of OLFM4, MSLN, ADH1A, ZG16, ST6GALNAC1, KRT23, FCGBP, CNTNAP2, KLK6, CA4, REG4, CA1, GCNT3, ITLN1, IL8, HLA-DRB1, LOC652775, SPINK4, CLCA1, and LYZ, while in another embodiment the one or more of the differentially expressed genes are selected from the group consisting of REG4, CA1, GCNT3, ITLN1, IL8, HLA-DRB1, LOC652775, SPINK4, CLCA1, and LYZ.

Analysis of the differentially expressed genes by various techniques identified a number of genes that were particularly informative with regard to whether or not the subject had progressed to stage III rectal cancer. It may therefore be preferable in some embodiments to determine the expression level of one or more of these genes. For example, in one embodiment, the one or more differentially expressed genes are selected from the group consisting of genes for interleukin-8,3-hydroxy-3-methylglutaryl coenzyme A synthase, carbonic anhydrase, ubiquitin, and cystatin. In another embodiment, it may be preferable to determine the expression level of all of these particularly effective genes; i.e., the one or more differentially expressed genes evaluated include the genes for interleukin-8,3-hydroxy-3-methylglutaryl coenzyme A synthase, carbonic anhydrase, ubiquitin, and cystatin.

In other embodiments, the plurality of differentially expressed genes may share functional characteristics, be part of a canonical pathway, or be part of a network involving a particular gene. These shared traits can be identified by analysis of the genetic profile. For example, the genes can be evaluated by a computer program which algorithmically identifies shared traits through analysis of an existing database. An example of a computer program that can be used to carry out this type of analysis is the Ingenuity® program, provided by Ingenuity® Systems, which is available through the internet.

Function analysis of the expressed genes has shown a number of functional categories in which the differentially expressed genes can be included, such as cancer, reproductive system disease, dermatological diseases and conditions, gastrointestinal disease, cellular movement, respiratory disease, inflammatory disease, genetic disorders, immunological disease, organismal injury and abnormalities, inflammatory response, cellular growth and proliferation, neurological disease, cell-to-cell signaling and interaction, and renal and urological disease. The differentially expressed genes used to determine if a subject with rectal cancer has stage III rectal cancer can be selected from any one or more of these functional categories. For example, in one embodiment, the differentially expressed genes comprise genes known to be functionally associated with cancer, gastrointestinal disease, or cellular movement.

Canonical pathways are groups of genes that are involved in particular signaling and metabolic biochemical pathways. The differentially expressed genes can be found in a variety of canonical pathways, including the antigen presentation pathway, cytotoxic T lymphocyte-mediated apoptosis of target cells, allograft rejection signaling, OX40 signaling pathway, bile acid biosynthesis, communication between innate and adaptive immune cells, B cell development, retinol metabolism, metabolism of xenobiotics by cytochrome P450, graft-versus-host disease signaling, nitrogen metabolism, altered T cell and B cell signaling in rheumatoid arthritis, crosstalk between dendritic cells and natural killer cells, and Nur77 signaling in T-lymphocytes. The differentially expressed genes used to determine if a subject with rectal cancer has stage III rectal cancer can be selected from any one or more of these canonical pathways. For example, in one embodiment, the differentially expressed genes comprise genes known to be part of immune-related pathways.

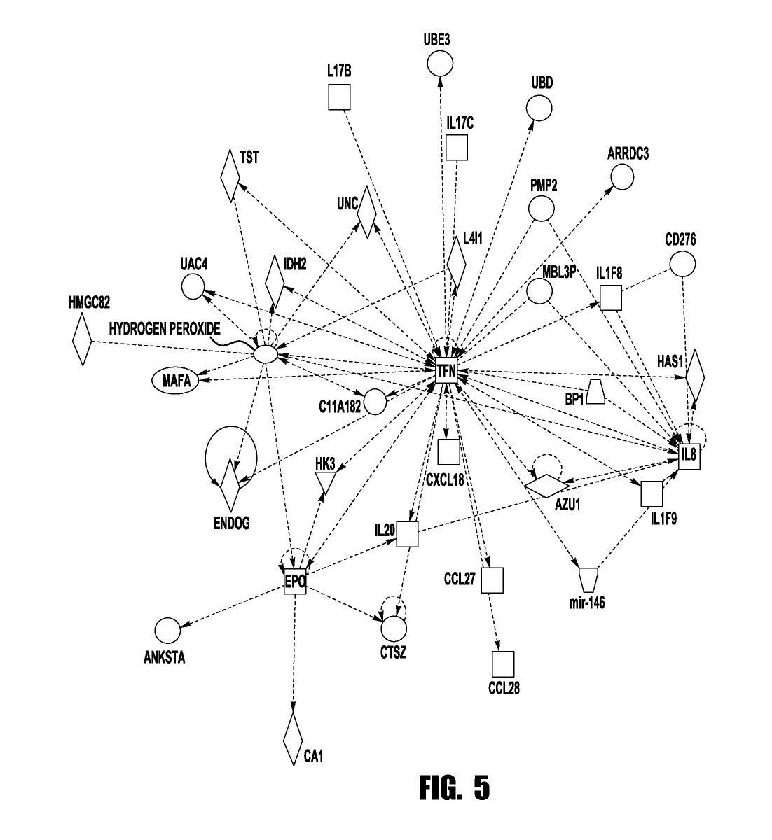

The differentially expressed genes can also be genes that are part of a network of genes. A network is a set of genes whose activity is interrelated based on the results from algorithmic analysis. Typically, a network includes a central gene to which numerous other genes show a degree of connection. An example of a gene network is shown in FIG. 5. Because numerous of the differentially expressed genes showing a high change in expression were shown to have an association with the tumor necrosis gene, in some embodiments the differentially expressed genes can be part of a network based on tumor necrosis factor.

The methods disclosed herein are useful for determining the risk that a subject diagnosed with rectal cancer has progressed to stage III rectal cancer. The risk that a subject has progressed to stage III rectal cancer refers to the probability that a subject has stage III rectal cancer. The risk that a subject has stage III rectal cancer can range from 0% to 100%, depending on the degree of changes in the expression level of the differentially expressed genes, the particular genes that have been evaluated, and in some embodiments the results from additional diagnostic methods.

The method of determining the risk that a subject diagnosed with rectal cancer has progressed to stage III rectal cancer can include the use of additional diagnostic methods beyond determining the expression levels of differentially expressed genes in order to obtain further data regarding cancer staging. Examples of additional methods that can be used include endorectal ultrasound and pelvic magnetic resonance imaging. These additional methods can be carried out using procedures known to those skilled in the art. The results of the additional methods can be factored into the overall diagnosis of whether or not the subject has progressed to stage III rectal cancer.

### Methods for Measuring Levels of Differentially Expressed Genes

The method described herein includes determining the expression levels of a plurality of differentially expressed genes. The method for determining the expression levels is not particularly limited, and all the gene detection methods known to those skilled in the art to which this invention pertains may be used. In some embodiments, the present methods may use real-time polymerase chain reaction (RT-PCR) to quantitatively measure gene expression by evaluating RNA levels obtained from rectal cancer tissue. Additional variants of RT-PCR technology, such those also including reverse transcription polymerase chain reaction, can also be used.

In other embodiments, the expression levels of a plurality of differentially expressed genes can be determined using a microarray. A microarray (more specifically, a DNA microarray) is two-dimensional array on a solid substrate (e.g., a glass slide or silicon thin-film cell) that assays large amounts of nucleic acid material using high-throughput screening methods. DNA microarrays can be used to measure the expression levels of large numbers of genes simultaneously or to genotype multiple regions of a genome. The DNA microarray includes numerous DNA spots, each of which contains a small quantity (i.e., picomoles) of a probe. These can be a short section of a gene or other DNA element that are used to hybridize a cDNA or cRNA sample from the subject under suitable stringency conditions.

The microarray includes probes that are immobilized in divided regions on a surface of a substrate at a high density. The microarray can include a plurality of probes, about 25 probes, about 50 probes, about 75 probes, or about 100 probes or more, or any numbers therebetween. The regions or spots can be arranged on the substrate at densities of, for example, 400/cm² or higher, 10³/cm², or 10⁴/cm². The substrate of the microarray is preferably coated with at least one activator selected from the group consisting of amino-silane, poly-L-lysine, and aldehyde, but is not limited thereto. In addition, the substrate may be at least one selected from the group consisting of silicon wafer, glass, quartz, metals, nylon films, nitrocellulose membranes, and plastics, but is not limited thereto. Probe-target hybridization is usually detected and quantified by detection of fluorophore-, silver-, or chemiluminescence-labeled targets to determine relative abundance of nucleic acid sequences in the sample.

A “Probe,” as used herein, refers to a polynucleotide molecule capable of hybridizing to a target polynucleotide molecule (e.g., mRNA transcribed from a differentially expressed gene). For example, the probe could be DNA, cDNA, RNA, or mRNA. A probe may be labeled, for example, with a fluorescent or radiolabel to permit identification. In one embodiment, a probe is of a sufficient number of base pairs such that it has the requisite identity to bind uniquely with the target and not with other polynucleotide sequences such that the binding between the gene expression product and the probe provides a statistically significant level of accurate identification of the differentially expressed gene. In one embodiment, the target is mRNA and the probe is a complementary piece of DNA or cDNA. In another embodiment, the target polynucleotide is cDNA or DNA and the probe is a complementary piece of mRNA or a complementary piece of DNA.

The term “hybridize” or “hybridizing” or “hybridization” refers to the formation of double stranded nucleic acid molecule between complementary sequences by way of Watson-Crick base-pairing. Hybridization can occur at various levels of stringency according to the invention. “Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to reanneal when complementary strands are present in an environment below their melting temperature. The higher the degree of desired homology between the probe and hybridizable sequence, the higher the relative temperature which can be used. As a result, it follows that higher relative temperatures would tend to make the reaction conditions more stringent, while lower temperatures less so. For additional details and explanation of stringency of hybridization reactions, see Ausubel, et al., Current Protocols in Molecular Biology, Wiley Interscience Publishers, (1995).

“Stringent conditions” or “high stringency conditions”, as defined herein, typically: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C. “Moderately stringent conditions” may be identified as described by Sambrook, et al., Molecular Cloning: A Laboratory Manual, New York: Cold Spring Harbor Press, 1989, and include the use of washing solution and hybridization conditions (e.g., temperature, ionic strength and % SDS) less stringent that those described above. An example of moderately stringent conditions is overnight incubation at 37° C. in a solution comprising: 20% formamide, 5×SSC (150 mM NaCl, 15 mM trisodium citrate), 50 mM sodium phosphate (pH 7.6), 5×Denhardt's solution, 10% dextran sulfate, and 20 mg/ml denatured sheared salmon sperm DNA, followed by washing the filters in 1×SSC at about 37-50° C. The skilled artisan will recognize how to adjust the temperature, ionic strength, etc., as necessary to accommodate factors such as probe length and the like.

Another method of determining the level of expression of a differentially expressed gene is to determine the level of protein product that is formed by the gene. Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified and/or quantified using chromatographic and electrophoretic techniques to achieve partial or complete purification (or purification to homogeneity). Analytical methods particularly suited to the preparation of a pure peptide are immunohistochemistry, ion-exchange chromatography, exclusion chromatography; polyacrylamide gel electrophoresis; isoelectric focusing. A particularly efficient method of purifying peptides is fast protein liquid chromatography or even HPLC.

In some embodiments, it may be preferable to also include a system (e.g., computer system and/or software) that is configured to receive data related to the expression levels of differentially expressed genes, and optionally other patient data (e.g., related to other staging information) and to calculate and display a risk score. In some such embodiments, the system employs one or more algorithms to convert the data into a risk score. In some embodiments, the system comprises a database that associates differentially expressed gene levels with risk profiles, based, for example, on historic patient data, one or more control subjects, population averages, or the like. In some embodiments, the system comprises a user interface that permits a user to manage the nature of the information assessed and the manner in which the risk score is displayed. In some embodiments, the system comprises a display that displays a risk score to the user.

Further, in one embodiment, the computer program is also capable of normalizing the patient's gene expression levels in view of a standard or control prior to comparison of the patient's gene expression levels to those of the patient population. In some embodiments, the computer is capable of ascertaining raw data of a patient's expression values from, for example, immunohistochemical staining or a microarray, or, in another embodiment, the raw data is input into the computer.

### Rectal Cancer

Cancer is a disease of abnormal and excessive cell proliferation. Cancer is generally initiated by an environmental insult or error in replication that allows a small fraction of cells to escape the normal controls on proliferation and increase their number. The damage or error generally affects the DNA encoding cell cycle checkpoint controls, or related aspects of cell growth control such as tumor suppressor genes. As this fraction of cells proliferates, additional genetic variants may be generated, and if they provide growth advantages, will be selected in an evolutionary fashion. Cells that have developed growth advantages but have not yet become fully cancerous are referred to as precancerous cells. Cancer results in an increased number of cancer cells in a subject. These cells may form an abnormal mass of cells called a tumor, the cells of which are referred to as tumor cells. The overall amount of tumor cells in the body of a subject is referred to as the tumor load. Tumors can be either benign or malignant. A benign tumor contains cells that are proliferating but remain at a specific site. The cells of a malignant tumor, on the other hand, can invade and destroy nearby tissue and spread to other parts of the body through a process referred to as metastasis. Rectal cancer is a subtype of colorectal cancer, and is a disease originating from the epithelial cells lining the rectum. The rectum extends from the upper end of the anal canal to the rectosigmoid junction of the gastrointestinal tract.

A method of characterizing the risk of developing stage III rectal cancer is described herein. The term “stage III” refers to the cancer stage of the rectal cancer. Cancer staging is the process of determining the extent to which a cancer has developed by spreading. The stage generally takes into account the size of a tumor, how deeply it has penetrated within the wall of an organ, whether it has invaded adjacent organs, how many regional lymph nodes it has metastasized to (if any), and whether it has spread to distant organs. Correct staging is critical because treatment (particularly the need for pre-operative therapy and/or for adjuvant treatment, the extent of surgery) is generally based on this parameter. Cancer staging, as used herein, includes re-staging, which is an additional determination of the stage of cancer after providing treatment.

Cancer staging involves assigning a number from I-IV to a cancer, with I being an isolated cancer and IV being a cancer which has spread to the limit of what the assessment measures. In general, Stage I cancers are localized to one part of the body. In rectal cancer, it is limited to the lining of the bowel wall. Stage II cancers are locally advanced with deepere penetration into the bowel wall. Stage III cancers may also be locally advanced, but in addition shows locoregional lymph node involvement. Stage 1V cancers are those that have metastasized, or spread to distant organs or throughout the body. While the present method provides a method for identifying subjects who have an increased risk of having stage III rectal cancer by determining the expression levels of differentially expressed genes, the present method can be combined with known methods for staging rectal cancer. Examples of such methods are described by Kim et al., RadioGraphics, 30, 503-516 (2010), the disclosure of which is incorporated by reference herein.

The presence of rectal cancer can be confirmed using a variety of techniques known to those skilled in the art. Symptoms of rectal cancer include worsening constipation, blood in the stool, weight loss, fever, loss of appetite, and nausea or vomiting in someone over 50 years old. Diagnosis of rectal cancer is generally obtained via tumor biopsy typically done during proctoscopy or colonoscopy. The extent of the disease is then usually determined by a CT scan of the abdomen and pelvis. There are other potential imaging tests such as PET, MRI, and endorectal ultrasound, which may be used in certain cases.

### Rectal Cancer Samples

The present method includes determining the expression levels of a plurality of differentially expressed genes in a rectal cancer sample from a subject that has been diagnosed with rectal cancer. A rectal cancer sample is a tissue sample obtained from a rectal cancer tumor, such as that obtained when a biopsy is carried out. The rectal cancer sample can be obtained using any typical biopsy method, such as excision, or a core needle biopsy or fine needle aspiration. Generally, the biopsy has a size ranging from 5 mm³ to a 1 cm³, although larger rectal cancer samples can be removed in some cases.

The expression levels of the differentially expressed genes can be determined either in vitro or ex vivo. In some embodiments, the subject has been diagnosed with stage II rectal cancer, while in other embodiments the subject has not been staged or has an earlier stage of rectal cancer. A rectal cancer sample may be fresh or stored. Rectal cancer samples may be or have been stored or banked under suitable tissue storage conditions. Preferably, rectal cancer samples are either chilled or frozen shortly after collection if they are being stored to prevent deterioration of the sample. In further embodiments, the rectal cancer sample can be a formalin-fixed, paraffin-embedded (FFPE) rectal cancer sample.

Any method known to one skilled in the art may be used to isolate the product of the differentially expressed genes (e.g., nucleic acid such as mRNA) from the rectal cancer sample of the subject. In some embodiments, the method further includes the step of extracting RNA from the rectal cancer sample before determining the expression levels of a plurality of differentially expressed genes. For example, an extract of the rectal cancer sample can be prepared, and then differential precipitation, column chromatography, extraction with organic solvent, and the like may be further performed. The nucleic acid isolated from the cells or tissues by the above method may be directly purified, or a predetermined region may be specifically amplified using an amplification method such as RT-PCR and separated. The nucleic acid includes mRNA, cDNA synthesized from mRNA, as well as DNA.

### Comparison of Differentially Expressed Gene Levels to Corresponding Control Values

A method of determining the risk that a subject that has been diagnosed with rectal cancer has stage III rectal cancer is described herein. The method includes comparing the expression levels of a plurality of differentially expressed genes selected from table 1a and/or table 1b in a rectal cancer sample from the subject to corresponding controls, and characterizing the subject as having an increased risk of having stage III rectal cancer if the expression levels of the genes from table 1a are increased compared to the corresponding control values, and/or the expression levels of the genes from table 1b are decreased compared to the corresponding control values. An increased risk refers to a higher percentage chance (e.g., 25%, 50%, or 75% chance) of having stage III rectal cancer in comparison to the normal risk that a subject who has been identified as having rectal cancer has progressed to stage III rectal cancer. The extent of the difference between the levels of the differentially expressed genes and their corresponding control values can be used to characterize the extent of the risk that the subject has stage III rectal cancer.

Comparison of each of the levels of the differentially expressed genes with a corresponding control value will provide difference value (e.g., fold change) for the particular differentially expressed gene being evaluated. By combining the difference values for a number of differentially expressed genes, one can obtain genetic profile score. Because the genetic profile score includes the differences of a number of different differentially expressed genes, it can provide a more accurate method for identifying whether a subject has an increased change of having stage III rectal cancer. Because the expression levels of differentially expressed genes can either increase or decrease in subjects with stage III rectal cancer, an overall score for the combined expression levels can be obtained by using absolute values. Alternately, the differentially expressed genes from tables 1a and 1b can be combined separately to obtain separate genetic profile scores for genes showing decreased and increased expression in stage III rectal cancer, respectively.

Control values are based upon the level of the differentially expressed genes in comparable rectal cancer samples obtained from a reference cohort. The reference cohort in this case is subjects who have been identified as having rectal cancer, but have not yet been diagnosed as having progressed to stage III rectal cancer. In some embodiments, the reference cohort can be a select population of human subjects.

The control value is preferably provided in a manner that facilitates comparison with the level of the differentially expressed genes. In other words, it is preferable that the units used to represent the level of differentially expressed genes, if units are present, are the same units used for the control values. For example, it may be preferable to normalize the control values with the levels of expression of the corresponding differentially expressed genes. By “corresponding,” what is meant is that each differentially expressed gene has a “corresponding” control value for the same gene, e.g., the level of expression of the interleukin-8 gene has a corresponding interleukin-8 control value, the level of expression of the HMG-CoA synthase gene has a corresponding HMG-CoA synthase conrol, etc.

“Normalization” refers to statistical normalization. For example, according to one embodiment, a normalization algorithm is the process that translates the raw data for a set of microarrays into measure of concentration in each sample. A survey of methods for normalization is found in Sarkar et al., Nucleic Acids Res., 37(2), e17 (2009). For example, a microarray chip assesses the amount of mRNA in a sample for each of tens of thousands of genes. The total amount of mRNA depends both on how large the sample is and how aggressively the gene is being expressed. To compare the relative aggressiveness of a gene across multiple samples requires establishing a common baseline across the samples. Normalization allows one, for example, to measure concentrations of mRNA rather than merely raw amounts of mRNA.

The control value can take a variety of forms. The control value can be a single cut-off value, such as a median or mean. Corresponding control values for the expression level of differentially expressed genes can include, for example, mean levels, median levels, or “cut-off” levels, that are established by assaying a large sample of individuals and using a statistical model such as the predictive value method for selecting a positivity criterion or receiver operator characteristic curve that defines optimum specificity (highest true negative rate) and sensitivity (highest true positive rate) as described in Knapp, R. G., and Miller, M. C. (1992). Clinical Epidemiology and Biostatistics. William and Wilkins, Harual Publishing Co. Malvern, Pa., the disclosure of which is incorporated herein by reference. A “cutoff” value can be determined for each differentially expressed gene that is assayed.

In some embodiments, a predetermined value is used. A predetermined value can be based on the levels of differential gene expression in a rectal cancer sample taken from a subject at an earlier time. For example, a predetermined value may be obtained from a subject who is known to have stage I or stage II rectal cancer. Unlike control values, predetermined values can be individualistic and need not be based on sampling of a population of subjects.

### Therapeutic Methods

The method of identifying subjects having an increased risk of having stage III rectal cancer can also include proving subjects having an increased risk with anticancer therapy suitable for treatment of stage III rectal cancer. A review of current treatment methods for rectal cancer is provided in Kosinski et al., CA Cancer J Clin., 62(3), 173-202 (2012), this disclosure of which is incorporated herein by reference. Anticancer therapy can include surgery, radiation therapy, administration of a therapeutic agent, or a combination thereof. In some embodiments, levels of one or more differentially expressed genes are assessed at one or more time points following treatment to monitor the effectiveness of the therapy and, as desired, to alter the therapy accordingly (e.g., continue therapy, discontinue therapy, change therapy).

The staging tests described herein are useful for determining if and when aggressive anticancer treatment should be prescribed for a subject. For example, subjects with a significantly increased risk of having stage III rectal cancer could be characterized as those in need of more aggressive intervention such as treatment with concurrent chemotherapy and radiation therapy, etc.

In one embodiment, the method comprises recommending administration or administering to the subject identified as having an increased risk of having stage III rectal cancer a suitable anticancer agent. Examples of anticancer agents approved for treatment of rectal cancer by the FDA include Bevacizumab, Cetuximab, Fluorouracil, Irinotecan Hydrochloride, Panitumumab, Regorafenib, and Ziv-Aflibercept, or combinations thereof. Use of Fluorouracil, particularly in combination with the vitamin leucovorin is particularly preferred. A wide variety of anticancer agents together with their recommended dosages, pharmacology, and contraindications can be found in the most recent version of the Physician's Desk Reference (currently the 67th edition), which is incorporated herein by reference. The amount of anticancer compound that is administered and the dosage regimen depends on a variety of factors, including the age, weight, sex, and medical condition of the subject, the severity of the disease, the route and frequency of administration, and the particular compound employed. When combination therapy is desired, radioprotective agents known to those of skill in the art may also be used.

Anticancer agents can be administered in association with at least one pharmaceutically acceptable carrier, adjuvant, or diluent (collectively referred to herein as “carrier materials”) and, if desired, other active ingredients. The anticancer may be administered by any suitable route known to those skilled in the art, preferably in the form of a pharmaceutical composition adapted to such a route, and in a dose effective for the treatment intended. The active compounds and composition may, for example, be administered orally, intra-vascularly, intraperitoneally, or topically (e.g., rectally). Formulation in a lipid vehicle may be used to enhance bioavailability.

In a further embodiment, the method includes recommending and/or conducting a surgical intervention for the subject such as protectomy, which can be done via an abdominoperineal resection or low anterior resection. In some embodiments, chemotherapy may be used together with surgical intervention, as adjuvant therapy, when anticancer agents are administered after surgery, or as neoadjuvant therapy, when anticancer agents are administered before surgery.

Finally, methods of treatment can also include radiation therapy. The most common preoperative radiation therapy regimens for treatment of rectal cancer include short course and long course external beam radiotherapy. In short course, 5 daily doses of 5 gray (Gy) are administered, while in long course about 2 Gy are administered dialed for about 25 days. Other methods of radiation therapy, such as neoadjuvant treatment or combination treatment with chemotherapy, are also known to those skilled in the art.

### Kits

Another embodiment of the present invention provides a kit for predicting the risk of that a subject with rectal cancer will have stage III rectal cancer. In some embodiments, the kit provides the capability to analyze polynucleotides from a rectal cancer sample using the polymerase chain reaction. The kit may include primer sequences for the polynucleotide product of the differentially expressed gene and the reagents necessary for amplification. One skilled in the art can easily design the primer by using conventional primer selection software. The kit may further include any one selected from the reactive reagent group consisting of a buffer, reverse transcriptase for synthesizing cDNA from RNA, dNTPs and rNTP (premixing type or separate feeding type), labeling reagents, and washing buffer, which are used in hybridization.

In other embodiments, the present invention provides a microarray as a kit. The microarray provides the capability to readily evaluate the expression level of a plurality of differentially expressed genes to determine if a subject has stage III rectal cancer. In this case, the kit may include the probe and reagents necessary for hybridization. The reagent necessary for hybridization may include for example a hybridizing buffer. The nucleic acids may be amplified or may not be amplified. Therefore, the kit may further include a reagent necessary for amplifying the nucleic acid. The nucleic acid may be labeled with a detectable label. Examples of the detectable label as such may further include any one selected from the group consisting of streptavidin-like phosphatase conjugate, chemifluorescent, and chemiluminescent, and are not limited thereto.

The microarray provided as a kit can have any of the components described herein for use in a microarray. The microarray can include probes for any of the differentially expressed genes described in tables 1a and 1b. For example, the microarray can include probes having polynucleotide sequences complementary to a polynucleotide sequence of genes expressing interleukin-8,3-hydroxy-3-methylglutaryl coenzyme A synthase, carbonic anhydrase, ubiquitin, and cystatin. In other embodiments, the microarray can provide the capability of evaluating about 25, about 50, about 75, or 100 or more differentially expressed genes. For example, one embodiment of the microarray kit can include at least 50 polynucleotide probes having polynucleotide sequences complementary to the polynucleotide sequence of the corresponding differentially expressed genes from table 1a and/or table 1b.

A kit for determining the risk that a subject diagnosed with rectal cancer has progressed to stage III can also include corresponding controls for the differentially expressed genes and a package for the microarray and the controls. In a further embodiment, the kit includes instructions for using the kit to determining the risk that a subject diagnosed with rectal cancer has progressed to stage III. Instructions included in kits can be affixed to packaging material or can be included as a package insert. While the instructions are typically written or printed materials they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” can include the address of an internet site that provides the instructions.

An example has been included to more clearly describe a particular embodiment of the invention and its associated cost and operational advantages. However, there are a wide variety of other embodiments within the scope of the present invention, which should not be limited to the particular example provided herein.

### Example

### High-Throughput Arrays Identify Distinct Genetic Profiles Associated with Lymph Node Involvement in Rectal Cancer

With the use of a large population of rectal cancers, the inventors evaluated objective genetic differences in primary tumors with and without associated lymph node metastases and identified key differences between the 2 groups.

### Methods

Rectal Cancer Samples. The Cleveland Clinic Department of Colorectal Surgery maintains an institutional review board-approved clinically annotated database and biobank for patients with colorectal cancer. Tumor tissues were obtained through a dedicated tissue procurement team within the Department of Anatomic Pathology, snap frozen, and stored at 280° C. This bank was queried for patients with stage II or III rectal cancer who were treated by protectomy. Patients who received neoadjuvant chemoradiation were excluded to avoid the influence of treatment on gene expression. A gastrointestinal pathologist confirmed the histopathological diagnosis of each specimen independently. Specimens chosen for analysis contained at least 60% tumor cells. Charts were reviewed to validate the pathological stage. Basic demographic, clinical, and tumor characteristics were analyzed. Quantitative variables are summarized by mean±SD or median with interquartile ranges. Categorical variables are summarized by frequency. Demographic and tumor differences between stage II and III populations were assessed by use of the Chi-squared or Fisher exact probability test for categorical variables and Wilcoxon rank-sum test for quantitative variables.

RNA Isolation and Microarray. Total RNA was extracted from fresh-frozen tumor tissue with the RNAqueous Kit (Ambion, Austin, Tex.) as previously described by our group. Sanchez et al., Br J. Surg., 96, 1196-1204 (2009). In brief, frozen tissue blocks stored at −80° C. were macrodissected to eliminate as much normal tissue as possible, and then tumor tissue was sectioned on a cryostat into 8 to 12×10 μm thick shavings. The tissue was suspended in 800 mL of lysis/binding solution and homogenized by passing through an 18-gauge needle and syringe 10 times. Subsequent steps of sample processing were performed according to the manufacturer's protocol. The RNA was then subjected to DNase treatment by the use of TURBO DNA-free (Ambion, Austin, Tex.). RNA samples were quantified by optical density 260/280 readings by using a spectrophotometer. To ensure RNA quality, each specimen was run on a 1% agarose gel to ensure lack of degradation before being hybridized for the microarray. The RNA was then assayed for whole genome gene expression by using 48,701 transcript-specific sequences on the Illumina Human-6 Expression v2 BeadChip (Illumina, San Diego, Calif.) as previously described.7 In brief, 100 ng of total RNA was amplified by an in vitro transcription amplification kit (Ambion, Austin, Tex.) and hybridized to the platform using commercially available kits (Illumina, San Diego, Calif.). Illumina BeadStation 500 software was used for imaging and normalization of data.

Microarray Analysis. Gene expression data were generated on the IlluminaHuman-6 v2 microarray platform that contains 48,701 transcripts. The Illumina Human-6 v2 is a single-color bead chip with probes derived from the National Center for Biotechnology Information Reference Sequence database. Probe content is well annotated and widely accepted. Expression data from the microarrays were compiled by using Beadstudio (version 2) then imported into Chipster, an R-based software interface enabling bioinformatic appraisals, Quality control was conducted on all quantile-normalized data. Density and box plots demonstrated an identical distribution of expression values. Further quality control using a nonmetric multidimensional scaling (NMDS) was conducted demonstrating the relationship between chips in 2 dimensions. Preprocessing and filtering were conducted as follows. Nonchanging genes were filtered according to SD (i.e, those genes with the lowest SD differed least in expression between both groups). Of all genes 99.7% were excluded, thereby returning a spreadsheet containing the top 147 changing genes. Only genes distributed out with 3 SDs from the mean were retained.

The groups for comparison included stage II (n=55) and stage III (n=22) rectal adenocarcinomas. Specifically, the mean expression of genes in stage II rectal cancer specimens were compared with corresponding means in the stage III cohort. An empirical Bayes 2 group t test was used to compare groups. Smyth et al., Stat Appl Genet Mol Biol., 3, Article3 (2004). Finally, only genes differing with a p value of less than 0.04 were retained for the purposes of graphic illustration in the heat maps and dendrograms. The resultant spreadsheet was used to generate a heat map and dendrogram demonstrating differences in expression profiles. To deal with the multiple testing issue that arises when dealing with a large number of statistical tests, we incorporated the Statistical Analysis of Microarrays (SAM) (Tusher et al., Proc Natl Acad Sci USA., 98, 5116-5121 (2001)) and Reproducibility-Optimized Test Statistic (ROTS) (Elo et al., IEEE/ACM Trans Comput Biol Bioinform., 5, 423-431 (2008)) using a false discovery rate set between 0.5 and 0.1 for these analyses. These analyses both set false discovery rates and account for the hyperinflated type 1 errors that occur with high-throughput analyses.

Gene Function Analysis. A data set containing gene identifiers and corresponding expression values/scores was uploaded into the Web-based program, Ingenuity IPA. Each identifier was mapped to its corresponding object in Ingenuity's Knowledge Base. These molecules, called Network Eligible Molecules, were overlaid onto a global molecular network developed from information contained in Ingenuity's Knowledge Base. Networks of Network Eligible Molecules were then algorithmically generated based on their connectivity. A functional analysis identified the biological functions and/or diseases that were most significant to the data set. Molecules from the data set that were associated with biological functions and/or diseases in Ingenuity's Knowledge Base were considered for the analysis. Right-tailed Fisher exact probability test was used to calculate a p value determining the probability that each biological function and/or disease assigned to that data set is due to chance alone. A canonical pathway analysis was conducted to identify the most significant canonical pathways from the Ingenuity Knowledge Base. Molecules from the data set that were associated with a canonical pathway in Ingenuity's Knowledge Base were considered for the analysis. The significance of the association between the data set and the canonical pathway was measured in two ways. First, a ratio of the number of molecules from the data set that map to the pathway divided by the total number of molecules that map to the canonical pathway is displayed. Second, a Fisher exact probability test was used to calculate a p value determining the probability that the association between the genes in the data set and the canonical pathway is explained by chance alone.

### Results

Patient and Tumor Characteristics. Seventy-seven rectal adenocarcinomas were included in the analysis. Fifty-five tumors were stage II and 22 tumors were stage III according to American Joint Committee on Cancer TNM pathology staging. The stage III patients were slightly younger than the stage II patients. There were no statistical differences between the patient populations in other demographics and tumor characteristics as shown in Table 2.

TABLE 2
Patient and Tumor Characteristics of Study Population

| | Stage II | Stage III | p value |
|---|---|---|---|
| N | 55 | 22 | |
| Mean age (years) | 66 | 60 | 0.02 |
| Gender (male/female) | 43/12 | 15/7 | 0.53 |
| Mean distance from anal verge (cm) | 8.9 | 7.5 | 0.08 |
| Median tumor size (cm) | 4.5 | 4.5 | 0.10 |
| Mean # lymph nodes examined | 23.1 | 22.1 | 0.91 |
| Tumor Differentiation | | | 0.60 |
| Well | 5 (9%) | 1 (4%) | |
| Moderate | 42 (78%) | 16 (73%) | |
| Poor | 7 (13%) | 5 (23%) | |

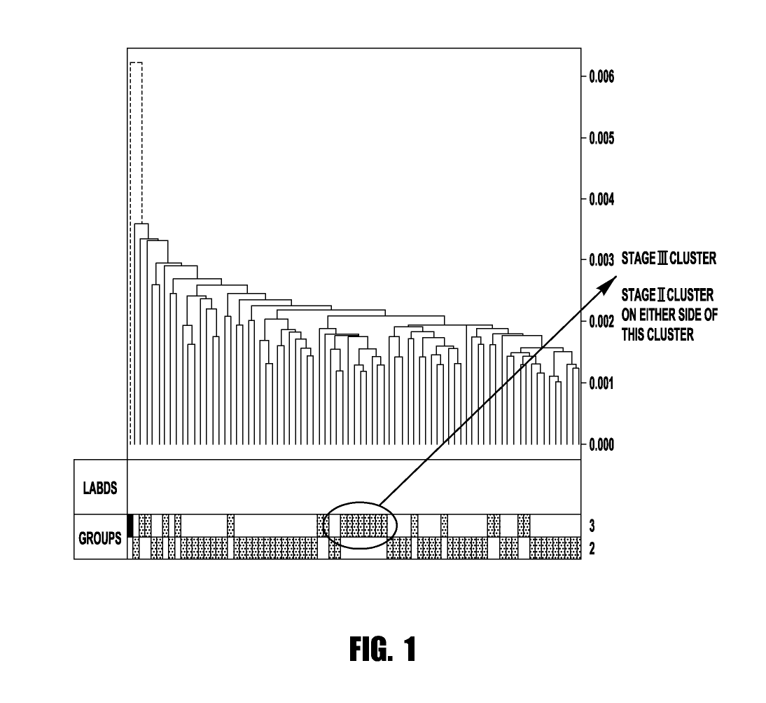

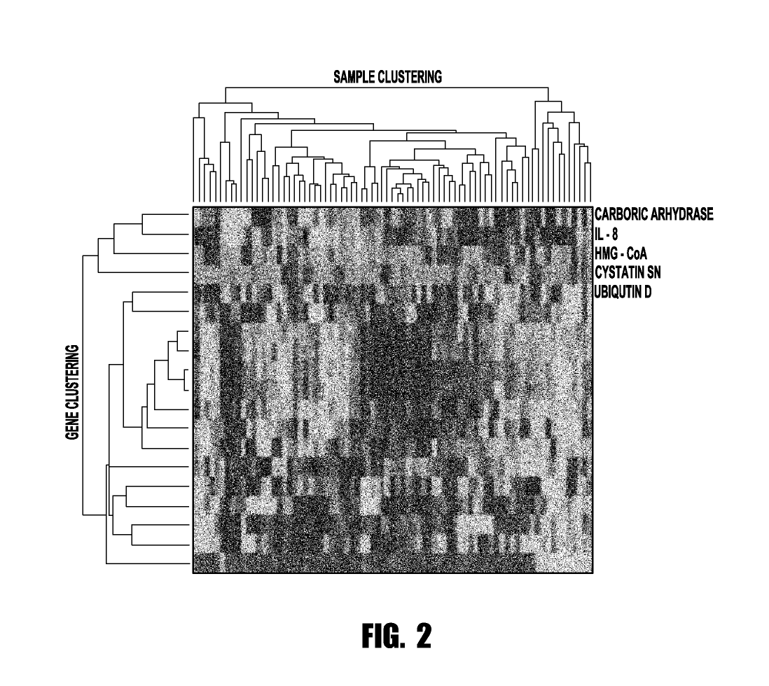

Gene Expression Patterns. To evaluate gene expression differences in the population, annotated samples were analyzed by NMDS in two dimensions. Nonmetric multidimensional scaling (NMDS) is a nonlinear mechanism of depicting variations according to gene expression. With the use of unsupervised clustering based on gene expression patterns, the samples tend to group together by stage. Two distinct, albeit somewhat overlapping, clusters emerged (data not shown). The clustering of samples is represented as a dendrogram in FIG. 1 and corresponds to the NMDS pattern. In the dendrogram, the component length of the vertical lines correlates with expression levels of genes, and the horizontal lines denote “relatedness” between samples. The majority of stage III rectal cancers centrally clustered together, whereas stage II cancers were more broadly distributed on either side of the middle stage III cluster. Filtering of genes yielded 147 top differentially expressed genes. Table 1 (provided earlier herein) depicts these top 147 differentially expressed genes between the clusters corresponding to stage II and stage III rectal tumors. The fold changes are the expression differences of each gene between stage II and stage III samples. A negative sign (2) before the fold change indicates that the gene is underexpressed in stage III relative to stage II tumors (shown in Table 1a), and a positive number in fold change indicates that gene is overexpressed in stage III relative to stage II tumors (shown in Table 1b). From these genes, only those with a p value of less than 0.04 on an empirical Bayes 2 group t test were used to generate a heat map with clustering as shown in FIG. 2. Again, two main clusters are readily apparent in the heat map and associated dendrograms. The dendrogram on the horizontal axis shows relatedness among the cancer samples. The dendrogram on the vertical axis shows relatedness among the genes. There were 12 tumors in the right-sided branch of the dendrogram cluster and 65 tumors in the left-sided cluster. Of the 12 clustered tumors, 11 (92%) were stage III rectal cancers. The one other tumor in this cluster was pathologically classified as a stage II tumor, but this patient experienced a recurrence of disease. Therefore, all tumors in this cluster were either stage III or developed a recurrent cancer. Of the 65 tumors in the left cluster, 54 (83%) were stage II and 11 (17%) were stage III. Looking at only the 55 stage II tumors, 54 (98%) were in this left cluster. The clustering according to stage was highly significant (p<0.0001). On reanalysis of the gene expression data with SAM and ROTS with the use of a false discovery rate of 0.1, similar clustering of samples based on stage was observed with significant overlap in outputs for each analysis (data not shown).

We next analyzed the clinical phenotype of the tumors within each cluster according to the development of recurrent disease. Looking specifically at the proportion of recurrence in both left and right groups, 24 of 65 (37%) tumors in the left cluster had recurrence, whereas 9 of 12 (75%) from the right cluster developed recurrence (p=0.024).

Among the top differentially expressed genes were interleukin-8 (IL-8), 3-hydroxy-3-methylglutaryl coenzyme A (HMG-CoA) synthase, carbonic anhydrase, ubiquitin, and cystatin (FIG. 2). We performed supervised clustering based on the expression of these five genes without regard for tumor stage. Again, two distinct clusters were readily evident as seen in FIG. 2. Four of these five genes were differentially upregulated in the cluster of 12 tumors that were predominantly stage III.

### Ingenuity IPA Gene Analysis

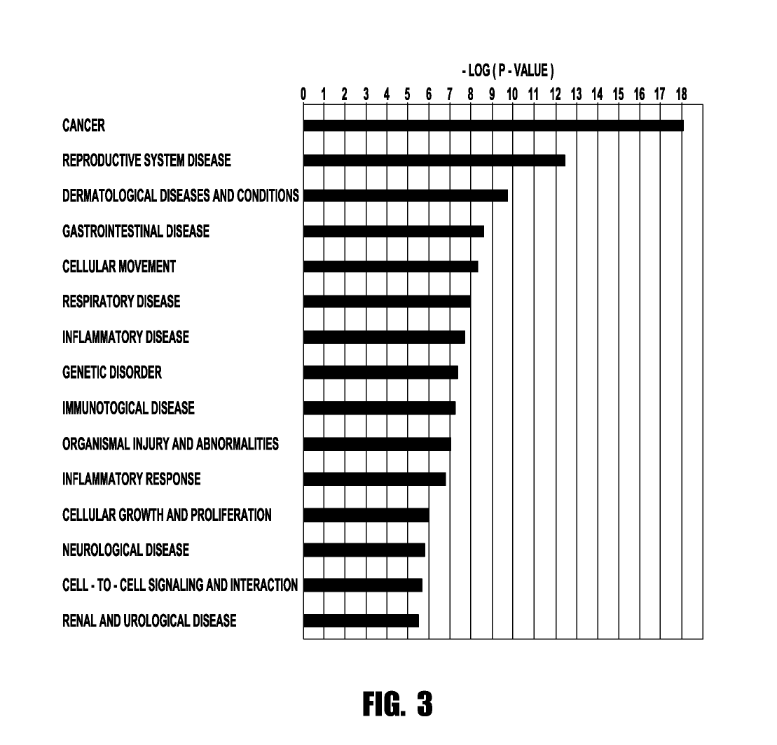

Gene Function Analysis. To investigate the biological functions involved in the 147 discriminating genes, Ingenuity IPA category analysis was performed. The top discriminating genes were associated with many different functions and biological processes. The associated 15 most represented functional categories and the number of genes in each category are shown in FIG. 3. The most common functional relationship was to cancer. Other key top functional pathways included gastrointestinal diseases and cellular movement.

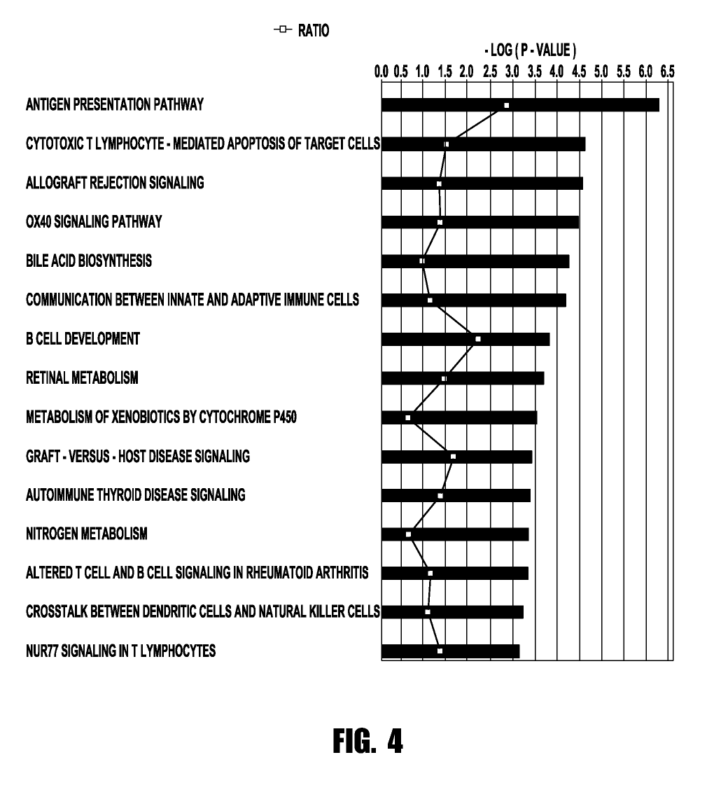

Gene Canonical Pathways. FIG. 4 displays the top canonical pathways represented by the top 147 differentially expressed genes. There was a predominance of immune-related canonical pathways represented.

Gene Network Pathways. A network is a graphical representation of the molecular relationships between molecules. Molecules are represented as nodes, and the biological relationship between 2 Ingenuity nodes is represented as an edge (line). All edges are supported by at least 1 reference from the literature, from a textbook, or from canonical information stored in the Ingenuity Knowledge Base. Four of the five top differentially expressed genes, marked by gray color, were mapped onto the same network, which was centered on tumor necrosis factor (FIG. 5).

### Discussion

This study used total genome cDNA microarray technology from a single-institution population to identify a gene signature associated with stage III rectal cancer. This is the first report of a genetic profile based solely on rectal cancers. The top differentially expressed genes are associated with key oncogenic pathways and may provide useful information toward understanding the biology of tumor progression.

Microarray technology has been used to examine differences in colorectal tumors with and without lymph node metastases in a few published studies. These studies have included mainly colon cancers with a small representation of rectal cancers. The assumption that colon and rectal cancers are interchangeable is untenable, because there are distinct biological differences between colon and rectal cancers and grouping both together will likely cloud the analysis. Wantanabe et al. (Dis Colon Rectum. 52, 1941-1948 (2009)) analyzed cDNA microarrays in 89 colorectal cancers in Japanese patients, of which only 22 were rectal cancers. They did not report how many of the rectal cancers were stage II or III. Similar to our results, the identified genes were associated with several key cancer-related pathways. Croner and colleagues reported gene expression differences in 80 colorectal cancers in a study from Germany. Croner et al., Ann Surg., 247, 803-810 (2008). There were 16 stage I/II and 16 stage III rectal cancers in this population. An analysis of the small subset revealed additional differentially expressed genes that were unique to rectal cancers, suggesting that additional or different molecular processes may be occurring in rectal cancer lymph node metastasis compared with that for colon cancers.

The top differentially expressed genes in our study did not overlap with those reported in the above-mentioned studies. There are at least 2 possible explanations for this. First, our study population is purely comprised of rectal cancers without the diluting influence of colon cancer profiles. Second, colorectal cancer is heterogeneous, and there is probably a large variability between populations, particularly between Japanese and Western patients. Therefore, there may actually be different underlying biology in these different populations.

Our top differentially expressed genes between patients with and without lymph node metastases were associated with many functions related to cancer, including antigen presentation, immune-mediated cytotoxicity and apoptosis, and cellular growth and proliferation. Within the most extreme cluster that contained nearly all stage III cancers, 4 genes were consistently informative. Literature review of the top genes in our signature supports their role in colorectal cancer biology. IL-8 has been shown to be associated with aggressive and highly invasive human colon carcinoma cells. Wang et al., J Dig Dis., 11, 50-54 (2010). In addition, IL-8 and its receptor have been linked to epithelial-mesenchymal transition, an important component of the metastatic process. Bates et al., Exp Cell Res., 299, 315-324 (2004). Carbonic anhydrase expression correlates with poor prognosis in a variety of tumors including colorectal cancer. Kivela et al., World J. Gastroenterol., 11, 155-163 (2005). Its upregulation in tumors is felt to be related to hypoxia and influenced by the tumor pH. Ubiquitin is a small protein tag that directs intracellular protein trafficking and controls various cellular mechanisms. Increased expression of ubiquitin has been associated with cancer progression and predicts therapy failure in colorectal cancer. Yan et al., Br J Cancer, 103, 961-969 (2010). HMG-CoA synthase is expressed in liver and several extrahepatic tissues, including the colon, and has been found to be downregulated in colon and rectum tumors. Birkenkamp-Demtroder et al., Cancer Res., 62, 4352-4363 (2002). Further exploration of these specific genes and their protein products could help unravel the mechanism of tumor progression.

Interestingly, Ingenuity Network analysis linked four of our top five genes with tumor necrosis factor (TNF). Although TNF itself was not one of the top genes, the link to immune reaction is prominent. Immune response has a broad impact on tumor initiation and progression, and many of these effects are mediated by proinflammatory cytokines. Among these cytokines, the protumorogenic function of TNF is well established. The role of TNF as a regulator of tumor-associated inflammation and tumorigenesis makes it an attractive target for cancer treatment. Grivennikov S I, Karin M, Ann Rheum Dis., 70(suppl 1), i104-i108 (2011).

Although the primary objective of this study was to identify biological differences as clues to understanding lymph node metastasis in rectal cancer, there are obvious clinical implications of using this information toward a diagnostic application. Accurate detection and diagnosis of locoregional lymph node involvement of rectal cancer remains a challenging clinical dilemma. Preoperative neoadjuvant treatment decisions are based on clinical staging, of which imaging is the cornerstone. However, current techniques such as endorectal ultrasound and pelvic magnetic resonance imaging still are only approximately 70% to 80% accurate. Similarly, clinical staging determines the surgical approach to rectal cancer. Although most early-stage cancers could be successfully cured by local excision, the risk of lymph node involvement is still significant enough to warrant protectomy in the face of uncertain lymph node status. A more objective and more accurate diagnostic test such as a gene signature could be obtained from a preoperative biopsy and thus help individualize treatment decisions. Komori et al., Int J Oncol., 32, 367-375 (2008). This information would also be useful in the postoperative setting to assign prognosis. Even histopathological staging is limited by sampling error and the constraints of light microscopy. Mejia et al., Adv Clin Chem., 52, 19-39 (2010). This is supported by the fact that recurrence rates of lymph node-negative patients can be as high as 30% to 50%. Compton C C, Greene Fla., CA Cancer J. Clin., 54, 295-308 (2004).

The gene clusters in our study were informative for associations with stage II and stage III rectal cancers. Given a specific expression profile, 100% of those tumors were either stage III or recurrent stage II cancers. Furthermore, patients with that signature had nearly double the recurrence rate in comparison with the other cluster, suggesting a more aggressive biology. Similarly, the stage II cluster was fairly uniform and predictive. The current model suffers from sensitivity in the stage III group, given that only 50% of the stage III tumors in the study had that expression pattern. That is not surprising given the heterogeneity and complexity of rectal cancer biology. Interestingly, the false-negative rate affects stage III and not stage II tumors, suggesting more homogeneity of gene expression in the early-stage group that is sufficiently strong to enable consistent correct identification of samples. We postulate that a core set of processes exists in stage II tumors that make their identification rather accurate. However, there are likely myriad and complex transcriptional profiles that may lead to lymph node metastases, and thus stage III tumor profiles are relatively less predictable because more cellular processes become uncontrolled. Thus, the heterogeneity of transcriptional profiles in the stage III group cannot be explained by only a small core of transcriptional events, and thus not all stage III tumors can be identified by a gene profile with only a few genes. As we increase the number of genes included in a classifier signature, the accuracy of sample classification improves to near 90%. We are in the process of developing and validating such a classifier.

A limitation to this study is potential type 1 errors that occur with high-throughput statistical analyses. When highly stringent false discovery rate testing such as Bonferroni, Benjamini-Yakutieri, and Benjamini-Hochberg were applied, the gene differentials did not reach statistical significance. However, both SAM and ROTS do account for false discovery rate, and these analyses yielded consistent results across platforms. We acknowledge that validation of individual gene expression levels by reverse transcriptase polymerase chain reaction would be required before any clinical application.

### CONCLUSIONS

Evaluation of total genome gene expression patterns between different clinical phenotypes provides a broad approach to identifying important aspects of a complex process. The information learned from this study reveals that there are distinct processes involved in the escape of tumor cells to lymph nodes specific for rectal cancer. Further exploration has great potential to learn about the biology of rectal cancer, but also provides the framework to build diagnostics tests to facilitate more individualized care.

The complete disclosure of all patents, patent applications, and publications, and electronically available material cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. A method of determining the risk that a subject that has been diagnosed with rectal cancer has stage III rectal cancer, comprising:
determining the expression levels of a plurality of differentially expressed genes selected from table 1a and/or table 1b in a rectal cancer sample from the subject,
comparing the expression levels of the plurality of genes with the corresponding controls, and
characterizing the subject as having an increased risk of having stage III rectal cancer if the expression levels of the genes from table 1a are increased compared to the corresponding control values, and/or the expression levels of the genes from table 1b are decreased compared to the corresponding control values.

2. The method of claim 1, wherein the plurality of differentially expressed genes each show an at least ±0.5 fold change relative to the corresponding control values.

3. The method of claim 1, wherein one or more of the differentially expressed genes are selected from the group consisting of SSBP1, HMGCS2, CEL, CST1, LY6G6D, SNAR-A1, DES, PCP4, ACTG2, and MYH11.

4. The method of claim 1, wherein one or more of the differentially expressed genes are selected from the group consisting of REG4, CA1, GCNT3, ITLN1, IL8, HLA-DRB1, LOC652775, SPINK4, CLCA1, and LYZ.

5. The method of claim 1, wherein the one or more differentially expressed genes are selected from the group consisting of genes for interleukin-8,3-hydroxy-3-methylglutaryl coenzyme A synthase, carbonic anhydrase, ubiquitin, and cystatin.

6. The method of claim 1, wherein the one or more differentially expressed genes comprise the genes for interleukin-8,3-hydroxy-3-methylglutaryl coenzyme A synthase, carbonic anhydrase, ubiquitin, and cystatin.

7. The method of claim 1, wherein the expression levels of the plurality of differentially expressed genes are determined using a microarray.

8. The method of claim 1, wherein the expression levels of at least 50 differentially expressed genes are determined.

9. The method of claim 1, wherein the plurality of differentially expressed genes are part of a network of genes based on tumor necrosis factor.

10. The method of claim 1, wherein the differentially expressed genes comprise genes known to be functionally associated with cancer, gastrointestinal disease, or cellular movement.

11. The method of claim 1, wherein the differentially expressed genes comprise genes known to be part of immune-related pathways.

12. The method of claim 1, wherein the subject has been previously diagnosed as having stage II rectal cancer.

13. The method of claim 1, further comprising the step of extracting RNA from the rectal cancer sample before determining the expression levels of a plurality of differentially expressed genes.

14. The method of claim 1, wherein the subject is a human.

15. The method of claim 1, further comprising the step of treating or recommending treatment of a subject identified as having an increased risk of having progressed to stage III with anticancer therapy suitable for treatment of stage III rectal cancer.

16. A microarray for determining the risk that a subject diagnosed with rectal cancer has progressed to stage III, comprising at least about 25 polynucleotide probes having polynucleotide sequences complementary to the polynucleotide sequence of the corresponding differentially expressed genes from table 1a and/or table 1b.

17. The microarray of claim 16, wherein the polynucleotide probes comprise probes having polynucleotide sequences complementary to a polynucleotide sequence expressed by the genes for interleukin-8,3-hydroxy-3-methylglutaryl coenzyme A synthase, carbonic anhydrase, ubiquitin, and cystatin.

18. A kit for determining the risk that a subject diagnosed with rectal cancer has progressed to stage III, comprising the microarray of claim 16, corresponding controls for the differentially expressed genes and a package for the microarray and the controls.

19. The kit of claim 18, wherein the kit further comprises instructions for using the kit to determining the risk that a subject diagnosed with rectal cancer has progressed to stage III.

20. The kit of claim 18, further comprising reagents for amplification of nucleic acids and detectable labels.
